(19) United States
(12) Patent Application Publication
(10) Pub. No.: US 2025/0134707 A1
(43) Pub. Date: May 1, 2025

(54) PUMP ELASTOMER SEAL ENHANCEMENT FOR SURGICAL CASSETTES

(71) Applicant: Alcon Inc., Fribourg (CH)

(72) Inventor: Behzad HASHEMI, Irvine, CA (US)

(21) Appl. No.: 18/901,548

(22) Filed: Sep. 30, 2024

Related U.S. Application Data
(60) Provisional application No. 63/594,910, filed on Oct. 31, 2023.

(51) Int. Cl. A61F 9/00; A61F 9/007; A61M 1/00

(52) U.S. Cl. CPC A61F 9/0008; A61F 9/00736; A61M 1/72

(57) ABSTRACT

Some embodiments provide a surgical cassette designed for use in ophthalmic surgical procedures, which features a multiple chamber pump elastomer to seal multiple chambers within the device. The elastomer is comprised of a silicone rubber component that incorporates a plurality of grooves on its top face and seal beads on its lower portion. The grooves enable lateral deformation of the silicone rubber under compression, improving the seal of the multiple chambers and reducing or eliminating leak paths, thus enhancing the device's performance. The seal beads, strategically placed on the lower portion of the silicone rubber component, prevent separation and lifting of the pump elastomer at the corners by conforming to the geometry of the corners.

### CROSS-REFERENCE TO RELATED APPLICATIONS

This application claims the benefit of U.S. Provisional Application Ser. No. 63/594,910 (filed on Oct. 31, 2023), the content of which is incorporated herein by reference in its entirety.

### INTRODUCTION

Cataract surgery involves removing a cataractous lens and replacing the lens with an artificial intraocular lens (IOL). The cataractous lens is typically removed by fragmenting the lens and aspirating the lens fragments out of the eye. The lens may be fragmented using, e.g., a phacoemulsification probe, a laser probe, or another suitable instrument. During the procedure, the probe fragments the lens, and the fragments are aspirated out of the eye through, e.g., a hollow needle or cannula. Throughout the procedure, irrigating fluid is pumped into the eye to maintain an intraocular pressure (IOP) and prevent collapse of the eye.

During cataract surgery, a surgical cassette having one or more peristaltic and/or venturi pumps and one or more valve assemblies may be operably coupled with a fluidics module of a surgical console and used to facilitate the aspiration and irrigation functionalities described above. In general, the one or more valve assemblies of the surgical cassette are operable to control the application of pressure and vacuum generated by the one or more peristaltic pumps during the surgical procedure.

However, conventional surgical cassettes have a number of significant shortcomings including, for example, relatively tight sealing tolerances, relatively high torque required for valve operation, restricted flow capacity, an inability to maintain a secure connection with the surgical console, relatively high fabrication costs and complexity, and redundancy issues, among others.

### SUMMARY

One or more embodiments herein include a surgical cassette that includes a multiple chamber pump elastomer assembly designed to seal multiple chambers within the surgical cassette. In some embodiments, the surgical cassette is designed for use in ophthalmic surgical procedures. The surgical cassette features one or more chambers for storing fluid and a pump elastomer that effectively seals the chambers when fully compressed. In some embodiments, the surgical cassette includes one or more grooves positioned on the pump elastomer. The grooves facilitate lateral deformation (or lateral displacement) of a portion of the pump elastomer when under compression and engaged with the base. The presence of grooves is advantageous in reducing or eliminating potential leak paths for the fluid at the interface between the pump elastomer and the base, ensuring a more secure and efficient performance during ophthalmic surgical procedures.

Some embodiments include a surgical cassette with a multiple chamber pump elastomer that serves to seal multiple chambers via seal beads. In some embodiments, the surgical cassette includes a plurality of seal beads strategically positioned on a base of the pump elastomer. These seal beads fill potential leak paths for the fluid at the interface between the pump elastomer and the base when the pump elastomer is under compression. By filling these leak paths, the seal beads eliminate or reduce internal leakage between the chambers of the multiple chamber pump elastomer, ultimately enhancing the performance of the surgical cassette during ophthalmic surgical procedures.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIG. 1 illustrates a schematic of an exemplary ophthalmic surgical system, in accordance with some embodiments.

FIG. 2A is a back side isometric view of an example surgical cassette which may be operably coupled to a console of the ophthalmic surgical system of FIG. 1, in accordance with some embodiments.

FIG. 2B is a back side elevation view of the surgical cassette of FIG. 2A, in accordance with some embodiments.

FIG. 2C is a side cross-sectional view of a pump assembly for use with the surgical cassette of FIG. 2A, in accordance with some embodiments.

FIG. 2D is a side cross-sectional view of the pump assembly of FIG. 2C coupled with the surgical cassette of FIG. 2A, in accordance with some embodiments.

FIG. 2E is another side cross-sectional view of the pump assembly of FIG. 2C coupled with the surgical cassette of FIG. 2A, in accordance with some embodiments.

FIG. 3A is a top side isometric view of an exemplary pump assembly which may be operably coupled to a surgical cassette, such as the surgical cassette of FIG. 2A, in accordance with some embodiments.

FIG. 3B is a side cross-sectional view of the pump assembly of FIG. 3A, in accordance with some embodiments.

FIG. 3C is a side cross-sectional view of the pump assembly of FIG. 3A coupled with the surgical cassette of FIG. 2A, in accordance with some embodiments.

FIG. 3D is a side cross-sectional view of the pump elastomer of FIG. 3A coupled with the surgical cassette of FIG. 2A, in accordance with some embodiments.

FIG. 4A is a back side isometric view of a surgical cassette illustrating an exemplary pump base portion, in accordance with some embodiments.

FIG. 4B is a side cross-sectional view of the exemplary pump base portion of FIG. 4A, in accordance with some embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure will now be described in detail with reference to the drawings, which are provided as illustrative examples of the disclosure so as to enable those skilled in the art to practice the disclosure. Notably, the figures and examples below are not meant to limit the scope of the present disclosure to a single embodiment, but other embodiments are possible by way of interchange of some or all of the described or illustrated elements. Moreover, where certain elements of the present disclosure can be partially or fully implemented using known components, only those portions of such known components that are necessary for an understanding of the present disclosure will be described, and detailed descriptions of other portions of such known components will be omitted so as not to obscure the disclosure.

The present disclosure relates generally to ophthalmic surgical cassettes, components therefor, and methods of use thereof. During certain ophthalmic surgical procedures, surgical cassettes may be utilized to facilitate aspiration/suction and infusion/irrigation in connection with a patient's eye. Typically, such surgical cassettes include one or more pump assemblies that are configured to engage with roller pump heads on a surgical console for generating a source of flow. The pump assemblies may each include a pump elastomer disposed over and scaling a segmented fluid chamber or volume and which, when engaged with a roller pump head, causes fluid within the volume to be driven in either a first or second direction, thereby generating downstream pressure and/or vacuum.

In many current surgical cassette designs, the pump elastomer includes an annular silicone rubber material that is overmolded onto a rigid plastic substrate ring (e.g., a base of the pump elastomer). Yet, implementation of the rigid substrate rings and silicone rubber overmold results in sealing issues due to the fixed boundary conditions created by the substrate rings. Sealing issues lead to leak paths and compromised performance, which is discussed in detail below.

For example, some pump elastomers are integrated into an inseparable assembly (two-shot molding) of silicone rubber overmold and plastic substrate ring. Such two-shot designs are advantageous over previous one-shot designs. However, under compression, the elastomeric silicone rubber of existing designs may impede a sealing interface between different segments, or chambers, of the pump assembly. Further, under compression, the silicone rubber may not have the freedom to move in a lateral direction as a roller pump head passes there over. As a result, the silicone rubber may, under compression, fold at the corners of the pump elastomer and create a void that can act as a leak path.

Accordingly, some embodiments described herein provide a pump elastomer having one or more grooves formed in, for example, an outer surface of the pump elastomer, which provides enhanced sealing of the segmented fluid volumes underneath. Such grooves facilitate the displacement of silicone rubber, and other elastomeric materials, under compression. The displacement of the silicone rubber facilitates a no-leak seal, which is described in detail below. In some embodiments, a pump assembly includes a base that engages with the pump elastomer and includes seal beads. Adding seal beads to the base, in accordance with some embodiments, advantageously fill the corner voids that may cause leak paths, which is described in further detail below.

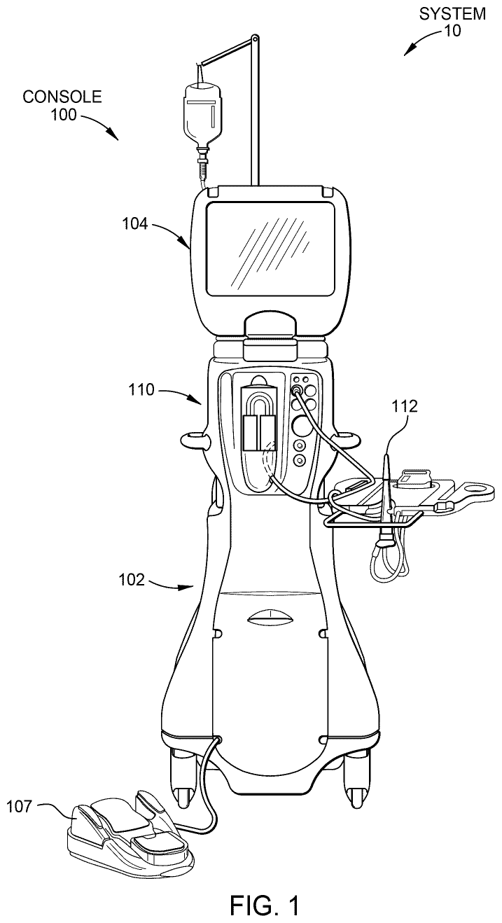

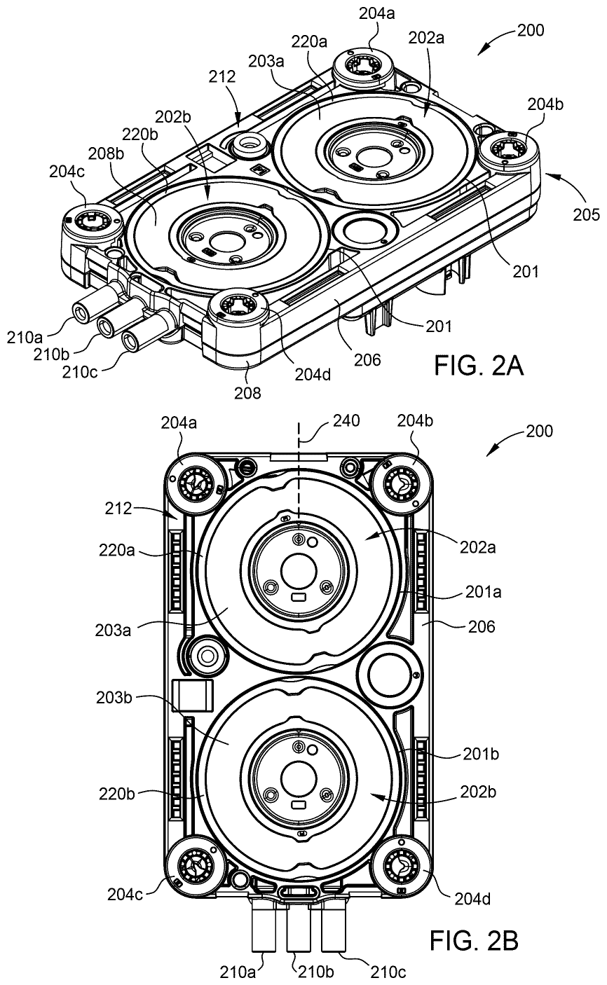

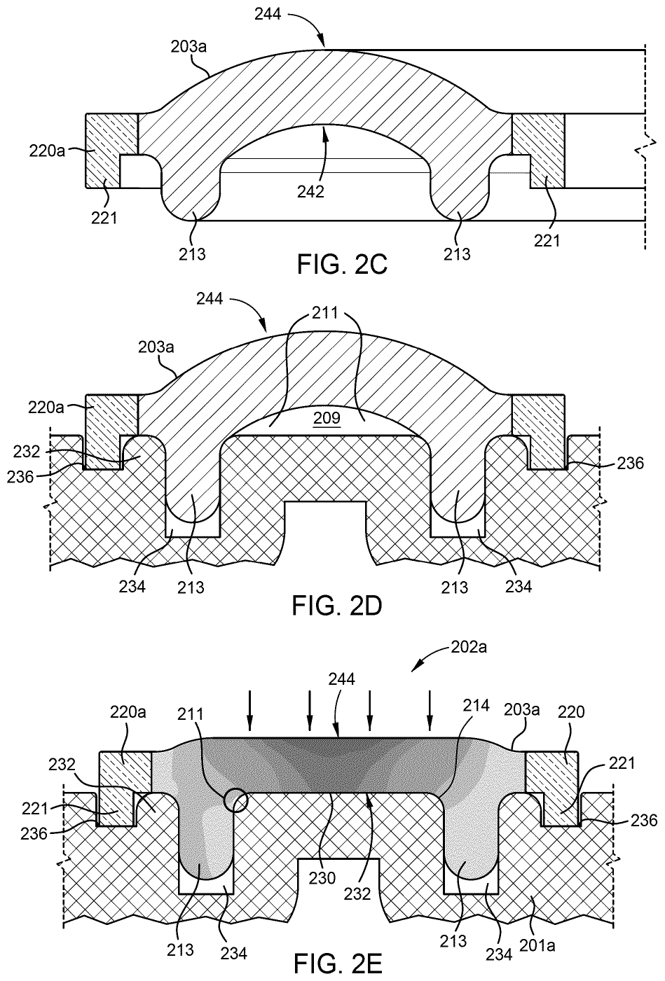

Referring now to FIGS. 1-2, FIG. 1 illustrates an example of an ophthalmic surgical system 10 that may be used to perform ophthalmic procedures on an eye, according to some embodiments. In the illustrated embodiments, system 10 includes console 100 (also referred to as a “surgical console”), interface device 107 (e.g., a foot pedal), and handpiece 112. Console 100 includes housing 102, which accommodates a computer and subsystems, fluidics subsystem 110, and display screen 104.

In some embodiments, handpiece 112 may be any suitable ophthalmic surgical instrument (e.g., an ultrasonically-driven phacoemulsification (phaco) handpiece, a laser handpiece, an irrigating cannula, a vitrectomy handpiece, or another suitable surgical handpiece). Fluidics subsystem 110 provides fluid control for one or more handpieces 112 (112a-c). For example, fluidics subsystem 110 may manage fluid for an irrigating cannula.

FIG. 2A-2B illustrate exemplary surgical cassette 200 for use with the embodiments described herein. Surgical cassette 200 may be operably coupled to a console of an ophthalmic surgical system (e.g., console 100 of ophthalmic surgical system 10 illustrated in FIGS. 1A-1B). FIG. 2A is a back side isometric view of surgical cassette 200, according to some embodiments. Meanwhile, FIG. 2B is a back side elevation view of surgical cassette 200 of FIG. 2A, according to some embodiments. FIGS. 2A-2B are described together herein for clarity.

Surgical cassette 200 includes two pump assemblies 202 (202a-b), which provide a source of pressure and/or vacuum and four valve assemblies 204 (204a-d), which control pressure and/or fluid communication within surgical cassette 200. In certain other embodiments, there may be only one pump assembly or more than two pump assemblies. In certain other embodiments, there may be more or less than four valve assemblies (e.g., two to six valve assemblies).

Surgical cassette 200 includes housing 205 having cassette base 206, cover assembly 208 coupled to cassette base 206, and inlet/outlet ports 210 (210a-c) in cassette base 206 that provide pressure and/or fluid communication between an internal environment and external environment of housing 205. Although not shown, flow lines (e.g., tubing) may be coupled between each port 210a-c and a corresponding component of fluidics subsystem 110 and/or corresponding handpiece 112 (shown in FIG. 1A).

In some embodiments, one of first pump assembly 202a or second pump assembly 202b provides a source of pressure (e.g., to create a driving force for fluid irrigation or infusion), while the other one of first pump assembly 202a or second pump assembly 202b provides a source of vacuum (e.g., to create a driving force for fluid aspiration or suction). First pump assembly 202a and second pump assembly 202b may include peristaltic pumps or any other suitable type of pump for generating pressure and/or vacuum. In some embodiments, first pump assembly 202a and second pump 202b assembly are identical to each other.

During certain ophthalmic surgical procedures, surgical cassette 200 plays a crucial role in facilitating aspiration, suction, infusion, or irrigation within a patient's eye. Pump assemblies 202a, 202b of surgical cassette 200 engage with roller pump heads in surgical console 100. The engagement of roller pump heads and pump assembly 202a, 202b generates a source of pressure and/or vacuum utilized in the surgical procedure. Pump assemblies 202a, 202b each include pump elastomer 203 (203a and 203b are shown, corresponding to each assembly 202a, 202b, respectively) that seals a fluid chamber between pump elastomer 203 and a portion of base 206. Hereinafter, the portion of base 206 that engages with each pump elastomer 203 is referred to as “base portion 201” (201a and 201b are shown, corresponding to each assembly 202a, 202b, respectively).

In some embodiments, the fluid chamber between each pump elastomer 203 and base 206 is segmented, and such segmentation may be provided by one or more features formed in pump elastomer 203. When engaged with a roller pump head, manual forces provided by rollers of the roller pump head against the pump elastomer drives fluid within the fluid chamber in either direction, generating downstream pressure or vacuum as needed.

As shown in FIGS. 2A-2B, in some embodiments, pump elastomer 203 may be annular, or ring-like, in shape, corresponding with a rotational path of the rollers of the roller pump heads of fluidics subsystem 110 of console 100. An annular shape of pump elastomer 203 allows the rollers of the roller pump heads to roll along, while pressing against, outer surface 244 of pump elastomer 203, thereby pushing fluid in one direction or the other and creating a vacuum or pressure force in the corresponding fluid line (e.g., 210a, 210b, and/or 210c) connected to pump assembly 202a, 202b. The coupling of each pump elastomer 203 to a substrate ring 220 (220a and 220b are shown in FIGS. 2A and 2B) is therefore advantageous for effective sealing. In some embodiments, pump elastomer 203 may be segmented with a monolithic piece or, in other embodiments, a wall of elastomer between the segments. Such segmentation ensures that each section functions independently while maintaining the overall integrity of the elastomer.

Valve assemblies 204 are disposed within cassette base 206. Valve assemblies 204 function cooperatively to control pressure and/or fluid communication within and through surgical cassette 200. Valve assemblies 204 may be operated to route fluid flow selectively between multiple channels of housing 205. Pump assemblies 202 and valve assemblies 204 are located on back side 212 of cassette base 206 which is visible in FIGS. 2A-2B. Surgical cassette 200 includes pump assembly 202a and 202b which are identical to one another.

Turning to FIGS. 2C and 2D, FIG. 2C depicts a side cross-section of pump assembly 202a when sectioned along line 240 of FIG. 2B, and FIG. 2D depicts a side cross-section of pump assembly 202a along line 240 when assembled with corresponding base portion 201a of cassette base 206. FIGS. 2C and 2D are described together herein for clarity.

As shown, pump assembly 202a comprises pump elastomer 203a. Pump elastomer 203a may be formed of any flexible and elastic polymer material suitable for use in ophthalmic pump systems. For example, pump elastomer 203 includes elastic properties that facilitate conforming to the contours of pump rollers heads as they roll along the pump elastomer 203a to drive fluid in the pump assembly 202a, thereby maintaining a secure and leak-free seal during use. In some embodiments, pump elastomer 203 may be made of silicone rubber and/or a fluorosilicone elastomer.

Pump elastomer 203a of pump assembly 202a comprises two seal extensions 213 that extend downwardly from opposing lateral sides of pump elastomer 203a. Seal extensions 213 are configured to engage with corresponding seal recesses 234 of base portion 201a to facilitate sealing of fluids between pump assembly 202a and base portion 201a. Similarly, substrate ring 220a comprises two ring extensions 221 that extend downwardly from substrate ring 220a. Ring extensions 221 are configured to bond with corresponding ring recesses 236 of base portion 201a (e.g., at the bottom of the ring extensions 221) to ensure secure and consistent placement of pump assembly 202a with base portion 201a. As an example, substrate ring 220a is configured to be ultrasonically welded to base 206, such that there may be a permanent weld joint at a surface of ring extensions 221 contacting a surface of ring recesses 236 of base portion 201a. By ultrasonically welding substrate ring 220a to base 206, pump elastomer 203a can be fixed to base 206.

In some embodiments, base portion 201a further includes plateau 230 disposed between seal recesses 234, and protrusions 232 disposed between seal recesses 234 and ring recesses 236. Together with an inner surface 242 of pump elastomer 203a, plateau 230 defines fluid chamber 209 through which fluid may be flowed, as driven by mechanical forces provided by rollers of a roller pump head against outer surface 244 of pump elastomer 203a, during use of surgical cassette 200. Fluid chamber 209 is generally sealed by the interaction of inner surface 242 of pump elastomer 203a against opposing edges 214 of plateau 230, and further by seal extensions 213 when disposed in seal recesses 234. During installation, when pump elastomer 203a is engaged with base portion 201a and is further compressed, i.e., pressed into base portion 201a, seal extensions 213 fill into seal recesses 234, creating a substantially permanent hermetic seal.

Plateau 230 serves as a sealing plane for fluid chamber 209 and constrains vertical displacement of pump elastomer 203a during operation. In certain embodiments, plateau 230 serves as a boundary (or wall) that pump elastomer 203a is configured to press against, thereby creating a seal. Positioning protrusions 232, as well as seal extensions 213, on opposite sides of plateau 230 further ensures that pump elastomer 203a remains properly aligned with base portion 201a during installation. That is, radial alignment may be made between seal extensions 213 and seal recesses 234.

FIG. 2E depicts side cross-section of Finite Element Analysis (FEA) simulation results of pump assembly 202a under a compression force against outer surface 244 (e.g., as supplied by rollers of a roller pump head). FEA is a numerical method used to simulate and analyze the behavior of materials, structures, and systems under various conditions, such as loading, temperature changes, or other external influences. FEA can be used to create detailed simulations and models of materials, structures, and/or systems (e.g., FIG. 2E).

In the context of pump assembly 202a, FEA can be employed to analyze stress distribution, deformation, and other mechanical properties to optimize the design and performance of the pump elastomer 203a. By simulating how the elastomeric material of pump elastomer 203a reacts to various forces and conditions, potential design modifications to improve the overall performance and reliability of the device can be realized. Thus, compression simulations aid to identify potential weaknesses in the design and areas that require optimization.

For example, pump elastomer 203a is coupled to substrate ring 220a and base 206. Substrate ring 220a is a rigid structure made from, for example, plastic, or, in some embodiments, metal alloys. Substrate ring 220a provides a firm and stable structure for pump elastomer 203a, anchoring it securely in place against base 206. The inherent rigidity of substrate ring 220a may restrict the lateral deformation (or lateral movement) of pump elastomer 203a. Thus, due to the coupling of substrate ring 220a and pump elastomer 203a, lateral deformation of the elastomeric material of pump elastomer 203a may be impeded by the rigidity of substrate ring 220a. When the roller pump head applies pressure on outer surface 244 of pump elastomer 203a, the elastomeric material of pump elastomer 203a is compressed and seeks to deform in response to the compression force. However, due to the tight coupling with substrate ring 220a, pump elastomer 203a may in certain examples, be restrained and struggle to easily move laterally or sideways.

The combination of substrate ring 220a, pump elastomer 203a, and corresponding features of base portion 201a creates a robust sealing mechanism for fluid chamber 209. Substrate ring 220a provides the necessary stability and rigidity, while pump elastomer 203a, through its elastic properties, can deform and adapt to the pressures applied during the operation of surgical cassette 200. Coupling pump elastomer 203a to substrate ring 220a is crucial in maintaining the overall integrity of pump elastomer 203a and preventing leaks and/or burst at high positive pressures. However, without features to facilitate lateral deformation under compression, pump elastomer 203a may be subjected to undue stress, potentially creating leak paths, which is discussed further below.

As shown in FIG. 2E, FEA simulations show that deformation of pump elastomer 203a is restricted by the fixed boundary condition on each side of pump elastomer 203a created by substrate ring 220a. Thus, substrate ring 220a prevents any material lateral deformation of pump elastomer 203a when pump elastomer 203a is under compression. Therefore, folds and/or lifts may form at edges 214 of plateau 230, causing separation and creating leak path 211 for fluid in fluid chamber 209, as shown in FIG. 2E. Accordingly, the embodiments described herein mitigate such restriction as caused by the fixed boundary conditions. For example, such restrictions may be mitigated with the implementation of grooves and/or seal beads, which is described in detail below.

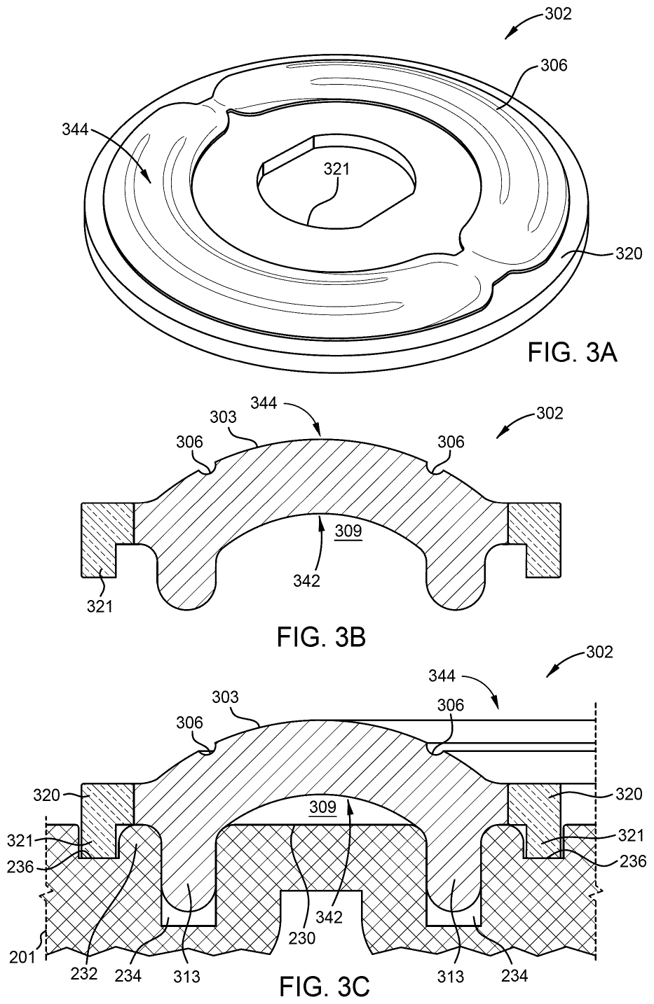

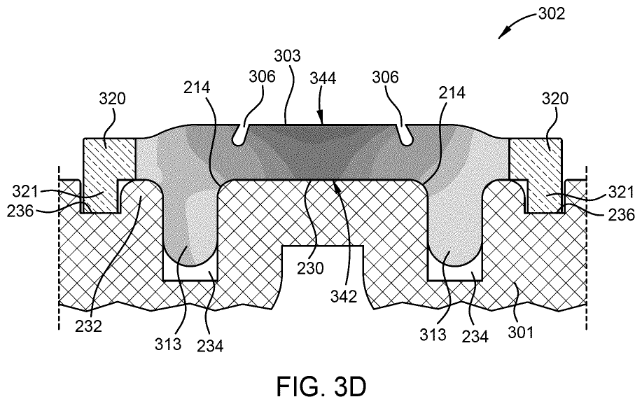

Referring now to FIG. 3A-3D, FIGS. 3A-3D depict an improved pump assembly 302, in accordance with one or more embodiments herein. In some embodiments, pump assembly 302 is configured for use with surgical cassette 200, or a surgical cassette similar thereto, that is utilized in ophthalmic surgical system 10, as discussed above. Accordingly, in certain examples, pump assembly 302 is depicted as assembled with base portion 201a.

Generally, pump assembly 302 may provide significantly improved performance and accuracy as compared to other pump assemblies. For example, pump assembly 302 may provide improved scaling of fluids flowing through one or more segments (e.g., chambers) of the pump assembly 302, thereby mitigating or eliminating any loss of internal pressure. Maintaining internal pressure ensures smooth and efficient fluid transfer (e.g., suction/aspiration or irrigation/infusion) during ophthalmic surgical procedures using system 10 and/or handpiece 112.

As shown in FIGS. 3A and 3B, in some embodiments, pump assembly 302 includes substrate ring 320 coupled to pump elastomer 303, which together are configured to engage with a base portion of a surgical cassette base (base portion 201a is shown). Pump elastomer 303 includes an outer surface 344 having grooves 306, and an inner surface 342 that, along with plateau 230 of base portion 201a, defines fluid chamber 309 through which fluid may be flowed, as driven by mechanical forces provided by rollers of a roller pump head against outer surface 344 of pump elastomer 303 during use of surgical cassette 200. Inner surface 342, along with plateau 230 and its edges 214, seals fluid chamber 309. In certain embodiments, fluid chamber 309 includes one or more segmented volumes disposed along a circumference of pump elastomer 303 and plateau 230, as formed by one or more features, such as walls, in pump elastomer 303 and/or base portion 201a.

Similar to pump elastomer 203a, pump elastomer 303 comprises two seal extensions 313 that extend downwardly from opposing lateral sides of pump elastomer 303. Seal extensions 313 are configured to engage with corresponding seal recesses 234 of base portion 201a to facilitate sealing of fluids between pump assembly 302 and base portion 201a. Similarly, substrate ring 320 comprises two parallel ring extensions 321 that extend downwardly from substrate ring 320. Ring extensions 321 are configured to bond with corresponding ring recesses 236 of base portion 201a (e.g., at the bottom of the ring extensions 321) to ensure secure and consistent placement of pump assembly 302 with base portion 201a. As an example, substrate ring 320 is configured to be ultrasonically welded to base 206, such that there may be a permanent weld joint at a surface of ring extensions 321 contacting a surface of ring recesses 236 of base portion 201a. By ultrasonically welding substrate ring 320 to base 206, pump elastomer 303 can be fixed to base 206.

Fluid chamber 309 is generally sealed by the interaction of inner surface 342 of pump elastomer 303 against opposing edges 214 of plateau 230, and further by seal extensions 313 when disposed in seal recesses 234. During installation, when pump elastomer 303 is engaged with base portion 201a and is further compressed, i.e., pressed into base portion 201a, seal extensions 313 fill into seal recesses 234, creating a substantially permanent hermetic seal. Plateau 230 serves as a sealing plane for fluid chamber 309, and constrains vertical displacement of pump elastomer 303 during operation.

Pump elastomer 303 may be formed of any flexible and elastic polymer material, suitable for use in ophthalmic pump systems and configured to facilitate a tight seal around fluids disposed in fluid chamber 309. For example, pump elastomer 303 includes elastic properties that facilitate conforming to the contours of pump rollers heads as they roll along the pump elastomer 303, thereby maintaining a secure and leak-free seal during use. In some embodiments, pump elastomer 303 may be made of silicone rubber and/or a fluorosilicone elastomer.

Pump elastomer 203 and pump elastomer 303 may be configured to serve the same function in surgical cassette 200, which includes creating a robust seal for fluid chambers within the surgical cassette 200, and to respond to the movement of the roller pump head to facilitate fluid flow during ophthalmic surgical procedures. For example, both pump elastomer 203 and pump elastomer 303 are annular or ring-like in shape for conforming to and aligning with a circular path of roller pump heads of fluidics subsystem 110 in surgical console 100. The annular shape allows for the roller pump heads to continuously and effective roll along the respective pump elastomer, pushing fluid in a desired direction and creating a vacuum or pressure in the fluid line connected to corresponding pump assembly. Pump elastomer 303, irrespective of the presence of grooves 306, is made from a flexible material like silicone rubber that can withstand repeated compression and release cycles as the roller pump head moves over outer surface 344. Flexibility of the pump elastomer 303 is advantageous, as flexing allows pump elastomer 303 to deform under pressure and then return to its original shape once the pressure is released.

A major difference between pump elastomer 203 and pump elastomer 303 is the presence of grooves 306 in the outer surface 344 of pump elastomer 303. The grooves 306 incorporated into the pump elastomer 303 design provide significant benefits in terms of scaling and device performance. The grooves 306 on the elastomer's surface facilitate lateral deformation of pump elastomer 303 under compression, thereby improving the sealing capabilities of the pump assembly 302 and overall device performance. For example, in response to compression forces applied by a roller pump head, pump elastomer 203, without grooves, can only be displaced vertically under compression, which can lead to increased stress at pump elastomer 203's interface with substrate ring 220 and potential leak paths. In contrast, pump elastomer 303, with grooves 306, facilitates lateral deformation of elastomeric material under compression, improving the seal and reducing or eliminating potential leak paths (e.g., 211).

Turning to FIG. 3D, during operation of pump assembly 302, pump elastomer 303 experiences compression, as caused by roller pump heads rolling along outer surface 344 of pump elastomer 303, to transfer fluids contained within fluid chamber 309 formed between the pump elastomer 303 and base portion 201a, and to create vacuum and/or pressure. Grooves 306 facilitate the lateral deformation of pump elastomer 303 under compression, thereby facilitating maintenance of a tight fluidic seal between base portion 201a and pump elastomer 303.

For example, when pump elastomer 303 is compressed due to the action of a roller pump head (e.g., FIG. 3D), the elastomeric material of pump elastomer 303 needs to displace in order to accommodate the applied compressive force(s). Grooves 306 provide channels or spaces (i.e., voids, trenches, and/or recesses) for the elastomer material to deform or expand into, enabling elastomeric material of pump elastomer 303 to displace laterally. Such lateral deformation enables pump elastomer 303 to maintain contact with base portion 301, improving the overall seal and reducing the chances of leaks at the interface between base portion 201a and pump elastomer 303.

In the absence of grooves 306, the elastomeric material would have limited space to move when compressed, which could lead to increased stress and potential leaks at the interface between pump elastomer 303 and base portion 201a. Grooves 306 effectively decrease the compression force required for lateral deformation of pump elastomer 303 compared to no grooves, while maintaining a robust seal with base portion 201a. By providing channels for lateral deformation, grooves 306 help maintain leak-free contact between pump elastomer 303 and base portion 201a.

Reducing or eliminating leak paths enhances the overall performance of surgical cassette 200 during surgical procedures. For example, reducing or eliminating leak paths ensures that intraocular pressure is maintained inside the eye of the patient, while at the same time providing smooth and efficient fluid flow during ophthalmic surgical procedures. As a result, safer and more effective outcomes for patients may be facilitated. Grooves 306 thereby contribute to enhanced fluidics control, increased device reliability, and extended device lifespan, ultimately leading to better patient care and more successful surgical outcomes.

In some embodiments, grooves 306 are advantageously positioned on outer surface 344 of pump elastomer 303, as shown in FIG. 3A. The depth, spacing, and/or arrangement of grooves 306 affect the effectiveness of grooves 306 in facilitating lateral deformation and improving the seal between base portion 201a and pump elastomer 303. Accordingly, in some embodiments, grooves 306 include a shape, depth, spacing, and/or arrangement that facilitate optimal lateral deformation of pump elastomer 303 under compression.

For example, grooves 306 are deep enough to allow for sufficient lateral deformation, but not so deep that they compromise the integrity or durability of pump elastomer 303. In some embodiments, a depth of grooves 306 may be substantially ranging between 0.1-4.0 mm (millimeters) (e.g., between 0.1-3.0 mm, 0.1-2.0 mm, or 0.1-1.0 mm), and a width of grooves 306 may be substantially ranging between 0.02-1.50 mm (e.g., between 0.03-1.40 mm, 0.04-1.30 mm, or 0.05-1.20 mm). In some embodiments, grooves 306 may include a depth of 0.64 mm, 1.0 mm, 1.5 mm, 2.0 mm, 2.5 mm, or 2.9 mm. In some embodiments, grooves 306 include a uniform depth, while in other embodiments, grooves 306 may include a tapered depth that begins flush and gradually deepens to the full depth.

In some embodiments, grooves 306 are advantageously positioned and sized for optimal lateral deformation of a particular elastomeric material of pump elastomer 303, such as silicone rubber. Optimal displacement may be determined via FEA simulations, discussed above. The spacing between grooves 306 ensures that grooves 306 effectively decrease stress and compression forces across pump elastomer 303. Such positioning of grooves 306 facilitates uniform and efficient lateral expansion, thereby providing no-leak seal between pump elastomer 303 and base portion 201a, as shown in FIG. 3D.

In some embodiments, the arrangement of grooves 306 may vary from what is shown in FIGS, 3A-3D depending on the specific design and application of the surgical cassette. For example, in some embodiments, grooves 306 may be arranged as concentric circles, as shown in FIG. 3A. In some embodiments, grooves 306 may be, parallel or skew line trenches, or other patterns selected for pump elastomer 303. The optimal arrangement of grooves 306 on pump elastomer 303 will depend on factors such as the geometry of the chambers within the surgical cassette (e.g., 200), the fluid dynamics within fluidics subsystem 110 of system 10, and other desired performance characteristics.

Thus, grooves 306 decrease stress and compression forces across pump elastomer 303, reducing the likelihood of localized stress concentrations. Such reduction may aid in prolonging a lifespan of pump elastomer 303 by minimizing wear and tear, ultimately extending the overall life of a surgical cassette (e.g., surgical cassette 200). Moreover, the enhanced scaling and reduced wear and tear provided by grooves 306 contribute to increased device reliability. Surgeons can have greater confidence in the performance and consistency of surgical console 100 during ophthalmic surgical procedures, which is crucial for ensuring patient safety and optimal surgical outcomes.

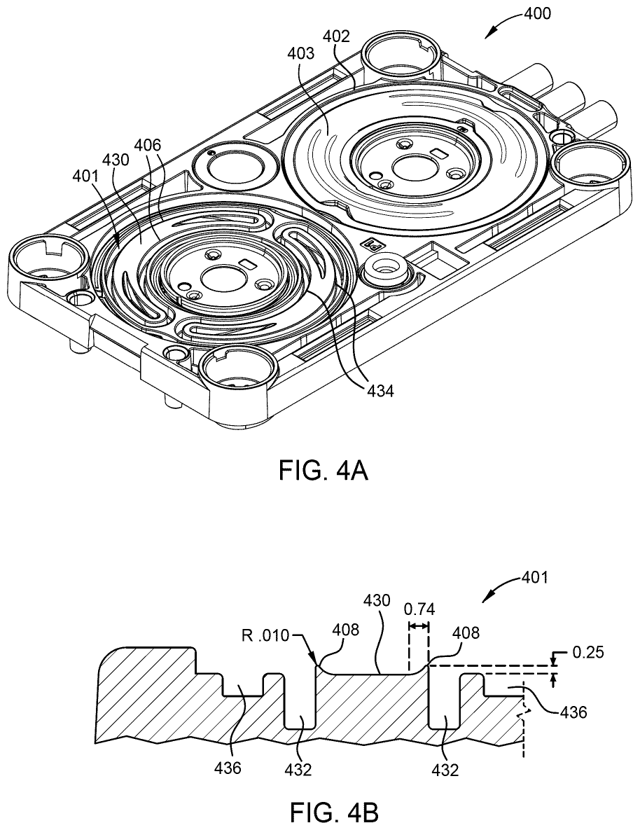

FIG. 4A depicts a back side isometric view of a surgical cassette 400 with one pump assembly 402 removed to reveal underlying base portion 401 of base 406, while another pump assembly 402 is coupled to the corresponding base portion 401. FIG. 4B illustrates a side cross-sectional view of the revealed base portion 401 of base 406. Accordingly, FIGS. 4A-4B are herein described together for clarity, in conjunction with FIGS. 1-3D.

Base portion 401 is an embodiment of base portions 201, 301, and similarly labeled parts and numbers thereof correspond to similar features having similar functionality. In some embodiments, base portion 401 includes features that enhance sealing and performance of the corresponding pump assembly. Base portion 401 is configured to interface with a pump elastomer of a pump assembly (e.g., pump elastomer 403 of pump assembly 402 shown in FIG. 4A) for containing, or scaling, fluid between base portion 401 and the pump elastomer 403. Pump elastomer 403 is an embodiment of pump elastomers 203, 303, and similarly labeled parts and numbers thereof correspond to similar features having similar functionalities.

In some embodiments, base portion 401 includes a plateau portion 430, seal beads 408 positioned on opposing sides of plateau portion 430, base recesses 434, ring recesses 436, and protrusions 432 (shown in FIG. 4B). Generally, seal beads 408 facilitate enhanced scaling capabilities and overall performance of pump assembly 402, and particularly, pump elastomer 403 of pump assembly 402.

In some embodiments, seal beads 408 may be advantageously positioned to fill (i.e., plug, obstruct) a leak path located at an interface between pump elastomer 403 and base portion 401 when coupled, thereby preventing the leaking of fluid from, for example, fluid chamber(s) (e.g., fluid chamber 309 in FIG. 3B) between the pump elastomer 403 and base portion 401 into a surrounding space. For example, seal beads 408 are configured to fill potential voids at the interface between the pump elastomer 403 and base portion 401 corresponding to leak paths (e.g., leak paths 211 in FIG. 2D) formed during compression of pump assembly 402.

Positioning seal beads 408 on base portion 401 is advantageous for maintaining a leak-free seal, ensuring smooth and efficient fluid flow during ophthalmic surgical procedures using surgical console 100. For example, seal beads 408 conform to the geometry of the corners at the interface between pump elastomer 403 and base portion 401 and prevent separation and lifting during pumping, thus enhancing the overall performance of surgical cassette 400 during surgical procedures. By anticipating such separation and lifting of the pump elastomer, seal beads 408 preemptively plug any potential leak paths (e.g., leak paths 211 in FIG. 2D) that could otherwise compromise the performance of surgical cassette 400.

As best seen in FIG. 4B, plateau portion 430 partially defines the fluid chambers or segments (e.g., fluid chambers 309 in FIG. 3B) between the pump elastomer 403 and base portion 401. Protrusions 432 may be positioned on opposite sides of plateau portion 430, as shown in FIG. 4B. Plateau portion 430 serves as a sealing plane that constrains vertical displacement of pump elastomer 403 during operation. Positioning protrusions 432 on opposite sides of plateau portion 430 ensures that pump elastomer 403 remains properly aligned with base portion 401 during installation. Base portion 401, in turn, features base recesses 434 that align with seal extensions (cf. seal extensions 313 in FIG. 3B) on pump elastomer 403.

During installation, when pump elastomer 403 is engaged with base portion 401 and is further compressed, i.e., pressed into base portion 201a, pump elastomer 403 seal extensions (e.g., 313) fill into base recesses 434, creating a permanent hermetic seal. Seal beads 408 are positioned on the base portion 401 to fill in any remaining gaps at the interface between pump elastomer 403 and the base portion 401, particularly at the edges of the plateau portion 430 (e.g., at the corners of the fluid chambers 309 when pump elastomer 403 is engaged with base portion 401), where potential leak paths may form during use. By conforming to the geometry of the pump elastomer 403, the corners of the fluid chambers 309, the seal beads 408 ensure a more comprehensive and robust seal, thereby enhancing the overall performance of the surgical cassette.

As described above, in some embodiments, base portion 401 may include seal beads 408. In some embodiments, seal beads 408 may include a height of substantially 0.25 millimeters (mm) and width of substantially 0.74 mm. In some embodiments, a radius of curvature (R) of seal beads 408 may be equal to substantially 0.10 mm. In some other embodiments, seal beads 408 may include a height, width, and radius of curvature less than, or more than 0.25 mm, 0.74 mm, and 0.10 mm, respectively.

In some embodiments, the height, width, arrangement, and shape of seal beads 408 may vary compared to the height, width, arrangement, and shape shown in FIGS. 4A-4B. For example, in some embodiments the height, width, arrangement, and shape of seal beads 408 may be selected to maximize their effectiveness in filling leak paths, preventing separation and lifting, and improving the sealing capabilities and overall performance of the surgical cassette. Because the height of seal beads 408 effects the ability to fill leak paths, such height is advantageously selected to conform to the corner geometry of pump elastomer 403 in order to fill leak paths and create an effective seal. Yet seal beads 408 do not interfere with or compromise the integrity of pump elastomer 403.

In some embodiments, seal beads 408 may be placed uniformly or in a pattern that maximizes engagement with a pump elastomer 403. For example, seal beads 408 may include a continuous ridge or unitary seal beads. In some embodiments, seal beads 408 may be organized in a linear, circular, or other patterns to ensure complete engagement with pump elastomer 403. The arrangement structure and pattern of seal beads 408 ultimately depend on the specific design of a pump elastomer and the presence and severity of any leak paths found during use, the geometry of the interface between the pump elastomer and the surgical cassette housing, and the desired sealing performance. Seal beads 408 therefore facilitate improved sealing by conforming to the geometry of leak path interfaces and create a more secure and effective seal for surgical cassette 400. The improved sealing capabilities directly impact the overall performance of the device, leading to more accurate and consistent fluid flow, which is essential for patient safety and surgical success.

Thus, by implementing one or more of the embodiments described herein, surgeons may have greater confidence in ophthalmic surgical systems (e.g., 10) performance and consistency during ophthalmic surgical procedures, which is crucial for ensuring patient safety and optimal surgical outcomes. The embodiments described herein provide a surgical cassette with an improved pump elastomer seals by modifying multiple chamber pump elastomers to enhance scaling and preventing such leaks. The grooves and seal beads design improvements, as well as the method for diagnosing and implementing these improvements, provide effective solutions for addressing the sealing issues present in traditional pump elastomer designs.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A surgical cassette, comprising:
a base;
one or more chambers storing a fluid;
a pump elastomer having a substrate ring and an elastomer seal, the pump elastomer being configured to seal the one or more chambers; and
one or more grooves positioned on the elastomer seal,
wherein the one or more grooves are configured to facilitate a lateral deformation of a portion of the elastomer seal when under compression and engaged with the base thereby reducing or eliminating a leak path of the fluid at an interface of the elastomer seal and the base.

2. The surgical cassette of claim 1, wherein the grooves have a depth ranging from 0.1 millimeter (mm) to 2.0 mm.

3. The surgical cassette of claim 1, wherein the elastomer seal comprises a silicone rubber.

4. The surgical cassette of claim 1, wherein the pump elastomer is a multiple chamber pump including at least two chambers.

5. The surgical cassette of claim 1, wherein the pump elastomer is configured to create a hermetic seal.

6. The surgical cassette of claim 1, wherein the grooves are uniformly spaced apart from each other, and wherein the grooves are positioned on a top face of the elastomer seal.

7. The surgical cassette of claim 1, wherein the grooves are configured to decrease stress and compression forces across the pump elastomer.

8. The surgical cassette of claim 1, wherein the grooves have:
a height ranging from 0.02 mm (millimeters) to 2.0 mm; and
a width ranging from 0.1 mm to 1.50 mm.

9. A surgical cassette, comprising:
a base;
one or more chambers storing a fluid;
a pump elastomer having a substrate ring and an elastomer seal, wherein the pump elastomer is configured to seal the one or more chambers; and
a plurality of seal beads positioned on the base,
wherein the plurality of seal beads are configured to fill a leak path of the fluid when the elastomer seal is under compression at an interface between the elastomer seal and the base, thereby eliminating or reducing the leak path and enhancing a performance of the surgical cassette.

10. The surgical cassette of claim 9, wherein the seal beads have a height ranging from 0.02 millimeter (mm) to 2.0 mm.

11. The surgical cassette of claim 9, wherein the seal beads have a radius of curvature (R) between 0.01 mm to 0.3 mm.

12. The surgical cassette of claim 9, wherein the seal beads are arranged in a pattern corresponding to one or more corners of the base.

13. The surgical cassette of claim 9, wherein the elastomer seal comprises silicone rubber.

14. The surgical cassette of claim 9, wherein the pump elastomer is a multiple chamber pump including at least two chambers.

15. The surgical cassette of claim 9, wherein the pump elastomer is configured to create a hermetic seal.
